(19) United States
(12) Patent Application Publication
(10) Pub. No.: US 2011/0086862 A1
(43) Pub. Date: Apr. 14, 2011

(54) POLYMORPHS OF PARDOPRUNOX

(76) Inventors: Jeroen Van Rheenen, Weesp (NL); Wilhelmus G.H.M. Muijselaar, Weesp (NL); Hendrik Teunissen, Weesp (NL)

(21) Appl. No.: 12/901,542

(22) Filed: Oct. 10, 2010

Related U.S. Application Data
(60) Provisional application No. 61/250,623, filed on Oct. 12, 2009.

(30) Foreign Application Priority Data
Oct. 12, 2009 (EP) 09 172802.2

(51) Int. Cl. A61K 31/496; C07D 413/10; A61P 25/16; A61P 25/18; A61P 25/22; A61P 25/24; A61P 25/00

(52) U.S. Cl. 514/254.02; 544/368

(57) ABSTRACT

This invention relates to a process for the preparation of 7-(4-methyl-1-piperazinyl)benzoxazol-2(3H)-one hydrochloride, a partial dopamine-D₂ receptor agonist and a full serotonin 5-HT_{1A} receptor agonist.

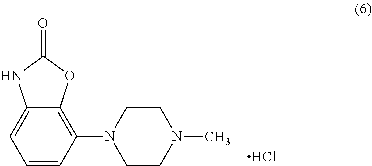

7-(4-methyl-1-piperazinyl)benzoxazol-2(3H)-one hydrochloride

The invention also relates to polymorphic forms of said compound, as well as to pharmaceutical compositions containing these compounds, to methods for preparing the compounds, to methods for preparing intermediates useful for their synthesis, and to methods for preparing compositions containing these compounds. The invention also relates to the use of such compounds and compositions, particularly their use in administering them to patients to achieve a therapeutic effect in conditions or diseases of the central nervous system, caused by disturbances of the dopaminergic and/or serotonergic systems, for example: anxiety disorders (including generalized anxiety, panic disorder and obsessive compulsive disorder), depression, autism, schizophrenia, Parkinson's disease, restless leg syndrome, and disturbances of cognition and memory.

This application claims the benefit of priority of EP 09 172802.2, filed Oct. 12, 2009, and U.S. Provisional Application No. 61/250,623, filed Oct. 12, 2009, the content of each of which is incorporated herein by reference.

Embodiments of the invention relate to the fields of pharmaceutical and organic chemistry. Embodiments of the invention relate to and provide processes for the preparation of 7-(4-methyl-1-piperazinyl)benzoxazol-2(3H)-one hydrochloride, a partial dopamine-D₂ receptor agonist and a full serotonin 5-HT_{1A} receptor agonist. Embodiments of the invention also relate to polymorphs of said compound, as well as to formulations containing and methods for using said compound.

The psychotropic piperazine derivative 7-(4-methyl-1-piperazinyl)benzoxazol-2(3H)-one mono-hydrochloride, also known as SLV308 and—recently—as pardoprunox, was first disclosed in WO 00/029397. The compound is a partial dopamine-D₂ receptor agonist and simultaneously a full serotonin 5-HT_{1A} receptor agonist. It is in clinical trials for the treatment for Parkinson's disease (R. Feenstra, et al., Drugs of the future, 26(2), 128-132, 2001).

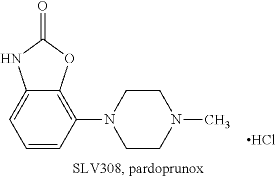

7-(4-methyl-1-piperazinyl)benzoxazol-2(3H)-one mono hydrochloride

Pardoprunox, mentioned in ‘example 2’ of WO 00/029397, is known as a hydrochloric acid salt. The synthetic route outlined in the patent has an acceptable yield, but it is not suited for synthesis on the scale required for a drug in clinical development, let alone the scale required for a commercially marketed drug. Problems with the original synthesis include: the use of bis-chloro-ethylamine, a suspected carcinogenic, the last intermediate is hard to process, and the end product contains a relatively large amount of impurities. A novel synthetic route to 7-(4-methyl-1-piperazinyl)benzoxazol-2(3H)-one mesylate was disclosed in WO 02/066449. Synthetic problems were overcome, but later it was decided to develop pardoprunox as a hydrochloric acid salt. The hydrochloric acid salt can be obtained by synthesizing the mesylate as described in WO 02066449, converting that to the free base, and preparing the hydrochloric acid salt from that.

### BRIEF DESCRIPTION OF DRAWINGS

FIG. 1. An XRPD pattern of the polymorphic form a of 7-[(4-methyl)-1-piperazinyl]-2(3H)-benzoxazolone hydrochloride.

FIG. 2. An IR (ATR) spectrum of the polymorphic form a of 7-[(4-methyl)-1-piperazinyl]-2(3H)-benzoxazolone hydrochloride.

FIG. 3. A Raman spectrum of the polymorphic form a of 7-[(4-methyl)-1-piperazinyl]-2(3H)-benzoxazolone hydrochloride.

FIG. 4. An XRPD pattern of the polymorphic form β of 7-[(4-methyl)-1-piperazinyl]-2(3H)-benzoxazolone hydrochloride.

FIG. 5. An IR (ATR) spectrum of the polymorphic form β of 7-[(4-methyl)-1-piperazinyl]-2(3H)-benzoxazolone hydrochloride.

FIG. 6. A Raman spectrum of the polymorphic form β of 7-[(4-methyl)-1-piperazinyl]-2(3H)-benzoxazolone hydrochloride.

### DESCRIPTION OF EMBODIMENTS OF THE INVENTION

Surprisingly, exploring experimental variations of synthesizing 7-(4-methyl-1-piperazinyl)-benzoxazol-2(3H)-one hydrochloride from its free base, two different polymorphs were discovered. The end product of one of the variants is the a-polymorph, while another variant yields the β-polymorph. Repeating the experimental conditions disclosed in WO 00/029397 proved that this route invariably leads to the β-polymorph.

Stability tests showed the a-polymorph to be more stable than the β-polymorph. For this reason, the α-polymorph is often used as an active ingredient in pharmaceutical compositions used to treat patients.

The α-polymorph can be obtained by dissolving 7-[(4-methyl)-1-piperazinyl]-2(3H)-benzoxazolone in a sufficient amount of a mixture of acetonitrile and water at reflux. Next, at reflux, HCl is added. Then, the mixture is cooled, and the product is isolated and washed. After drying to constant weight at elevated temperature and low pressure, the α-polymorph is obtained in a high yield.

The β-polymorph can be obtained by dissolving 7-[(4-methyl)-1-piperazinyl]-2(3H)-benzoxazolone in a sufficient amount of acetonitrile to obtain a clear solution at reflux. Next, at reflux, HCl is added. Then, the mixture is cooled, and the product is isolated and washed. After drying at elevated temperature and low pressure, the β-polymorph is obtained in a high yield.

The α-polymorphic form of 7-[(4-methyl)-1-piperazinyl]-2(3H)-benzoxazolone hydrochloride is defined by the following physicochemical characteristics:

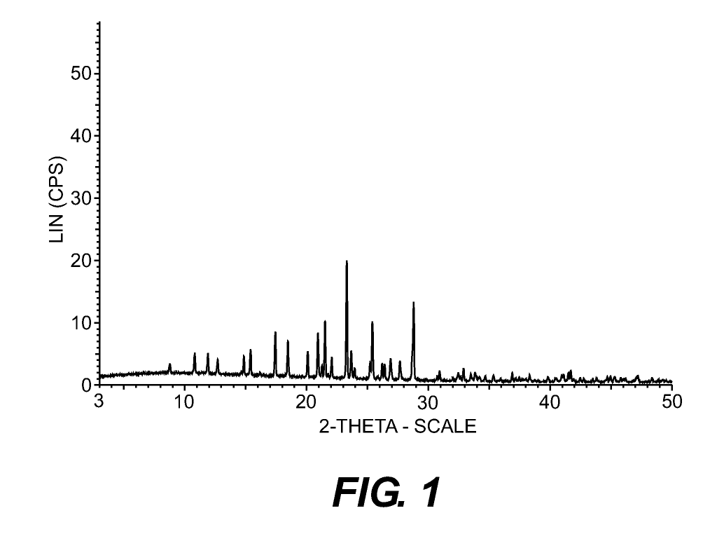

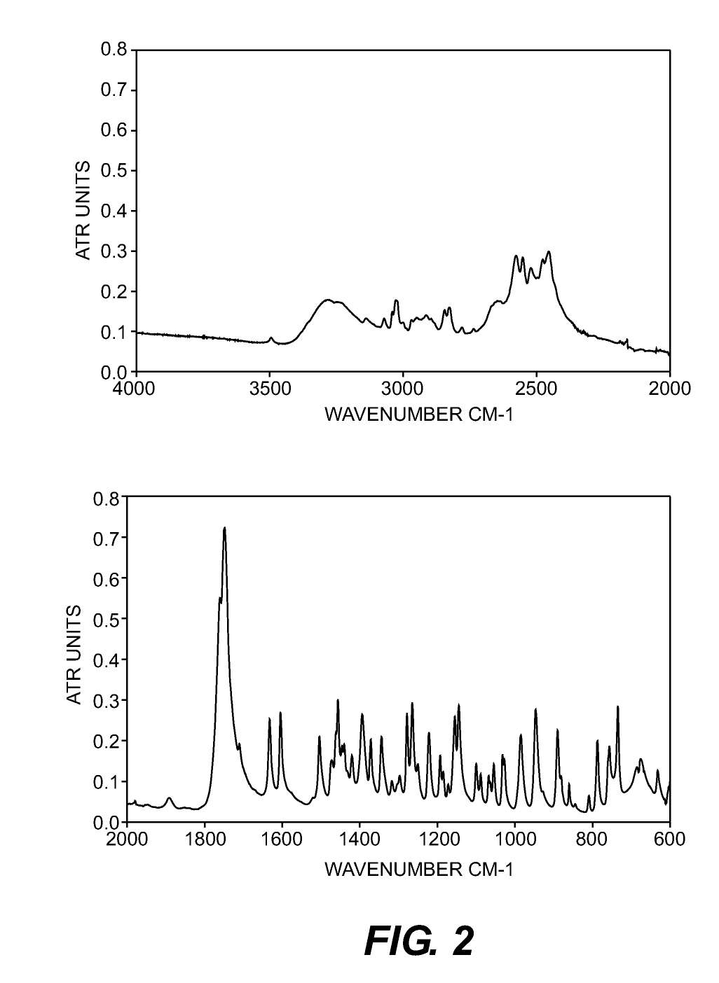

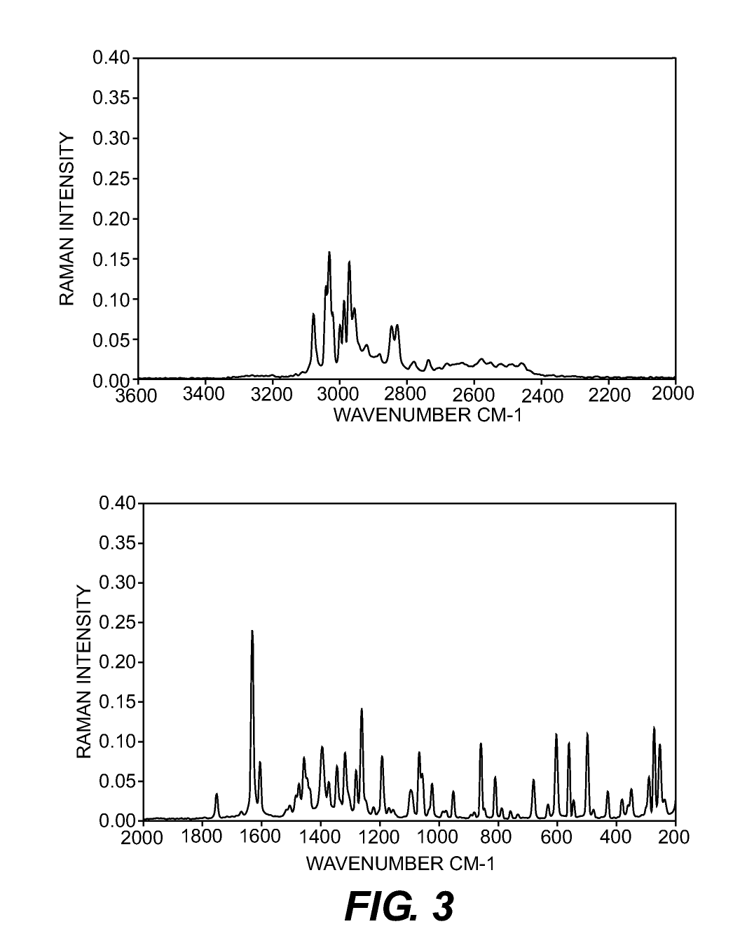

(i) An X-ray powder diffraction (XRPD) pattern having characteristic reflexes (expressed in degrees of diffraction angle 2θ) at about: 15.3, 17.4, 18.4, 20.1, 20.9, 21.5, 23.3, 23.6, 25.4, and 28.8. Diffraction angles are indicated as mean values (±0.1°) of six independent measurements. The complete XRPD pattern for the polymorphic form α is shown in FIG. 1. Most distinguishing peaks are those at about: 17.4, 21.5, 23.3 and 28.8.
(ii) An infrared (IR) spectrum recorded in attenuated total reflectance (ATR) having characteristic absorption bands expressed in reciprocal centimeters at about: 2454, 1749, 1632, 1604, 1456, 1394, 1265, 1144, 947, and 735. Absorption bands are indicated as mean values of six independent measurements. The complete IR spectrum for the polymorphic form a is shown in FIG. 2. Most distinguishing bands are those at about 2454 and 1604.
(iii) A Raman spectrum having characteristic absorption bands expressed in reciprocal centimeters at about: 3079, 3031, 2987, 2972, 1632, 1262, 859, 561, 499, and 273. Absorption bands are indicated as mean values of six independent measurements. The complete Raman spectrum for the polymorphic form α is shown in FIG. 3. Most distinguishing bands are those at about 3079, 3031 and 1632.

The β-polymorphic form of 7-[(4-methyl)-1-piperazinyl]-2(3H)-benzoxazolone hydrochloride is defined by the following physicochemical characteristics:

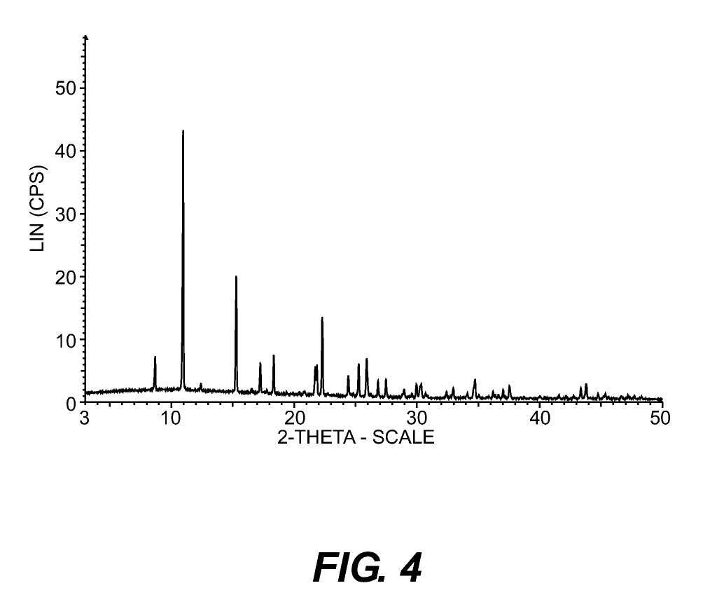

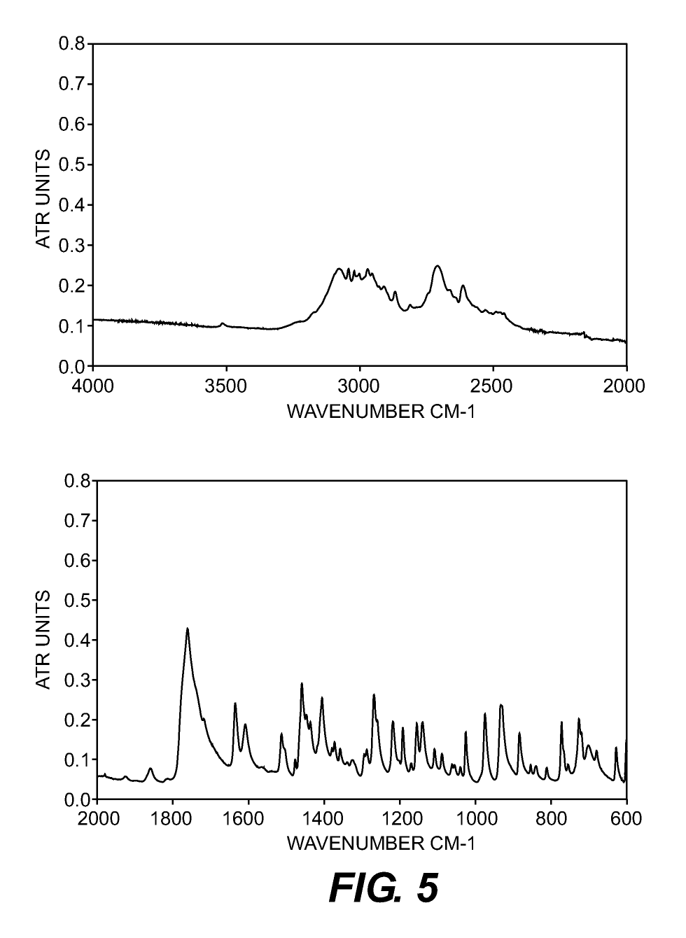

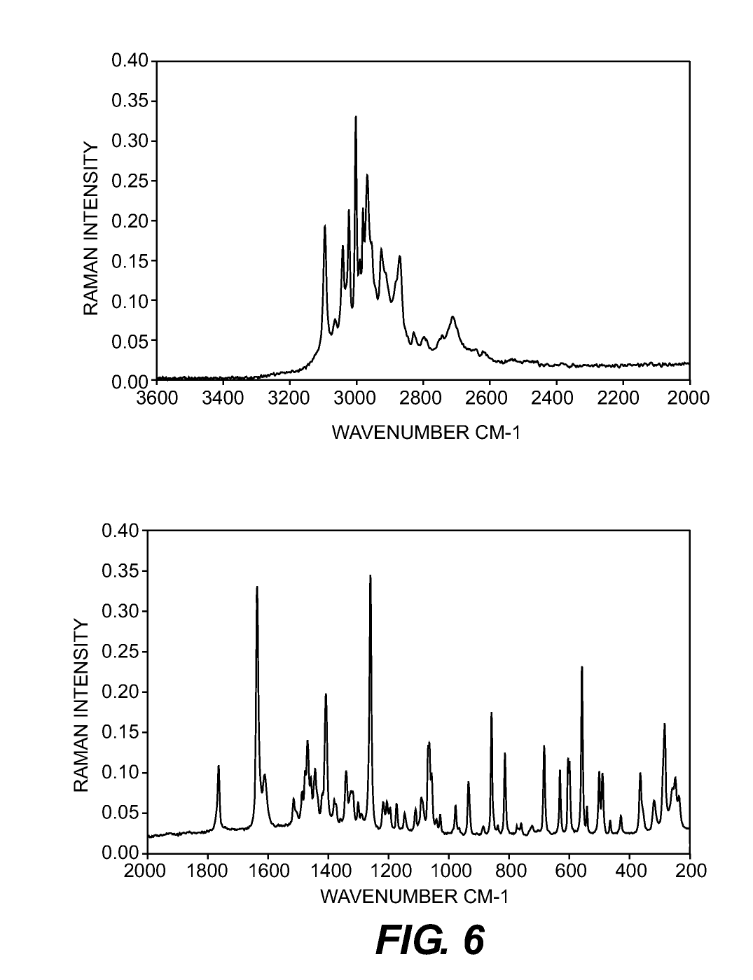

(i) An XRPD pattern having characteristic reflexes (expressed in degrees of diffraction angle 2θ) at about: 8.6, 10.9, 15.3, 17.2, 18.3, 21.7, 21.8, 22.3, 25.3, and 25.9. Diffraction angles are indicated as mean values (±0.1°) of six independent measurements. The complete XRPD pattern for the polymorphic form β is shown in FIG. 4. Most distinguishing peaks are those at about: 10.9, 15.3, 18.3 and 22.3.
(ii) An IR spectrum, recorded in ATR, having characteristic absorption bands expressed in reciprocal centimeters at about: 2709, 1761, 1635, 1459, 1405, 1268, 975, 930, 772, and 726. Absorption bands are indicated as mean values of six independent measurements. The complete IR spectrum for the polymorphic form β is shown in FIG. 5. Most distinguishing bands are those at about 2709 and 975.
(iii) A Raman spectrum having characteristic absorption bands expressed in reciprocal centimeters at about: 3095, 3023, 3002, 2968, 1636, 1408, 1260, 858, 558, and 284. Absorption bands are indicated as mean values of six independent measurements. The complete Raman spectrum for the polymorphic form β is shown in FIG. 6. Most distinguishing bands are those at about 3095, 3002 and 1408.

Single crystal X-Ray diffraction data for the crystal structure determination of polymorphic forms a and β of 7-[(4-methyl)-1-piperazinyl]-2(3H)-benzoxazolone hydrochloride are provided below.

Embodiments of the present invention also relate to 7-[(4-methyl)-1-piperazinyl]-2(3H)-benzoxazolone hydrochloride in which at least about 50 weight percent (wt. %) of the compound, for example, at least about 60 wt. % thereof, at least about 80 wt. % thereof, at least about 90 wt. %, or at least about 95 wt % of the compound, is in the polymorphic a form, and is substantially devoid of β polymorphic form thereof. Substantially devoid in the context of embodiments of the present invention means an amount of less than 10%, for example, less than 5% w/w. In some embodiments, at least about 99% wt.% of 7-[(4-methyl)-1-piperazinyl]-2(3H)-benzoxazolone hydrochloride is in the polymorphic α form.

Embodiments of the invention also relate to a process for the preparation of 7-[(4-methyl)-1-piperazinyl]-2(3H)-benzoxazolone hydrochloride, comprising:

(i) catalytic hydrogenation of 5-chloro-7-nitro-2(3H)-benzoxazolone (1) yielding 7-amino-2(3H)-benzoxazolone (2):

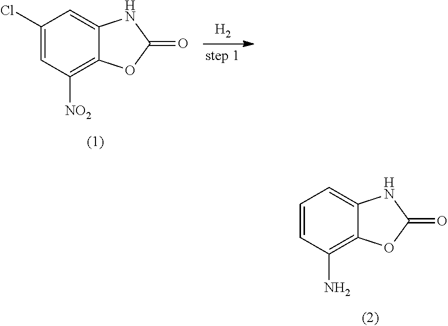

(ii) reacting 7-amino-2(3H)-benzoxazolone (2) with N-methyldiethanolamine (3) in the presence of methanesulphonic acid anhydride, to yield 7-[(4-methyl)-1-piperazinyl]-2(3H)-benzoxazolone methanesulfonate (4).

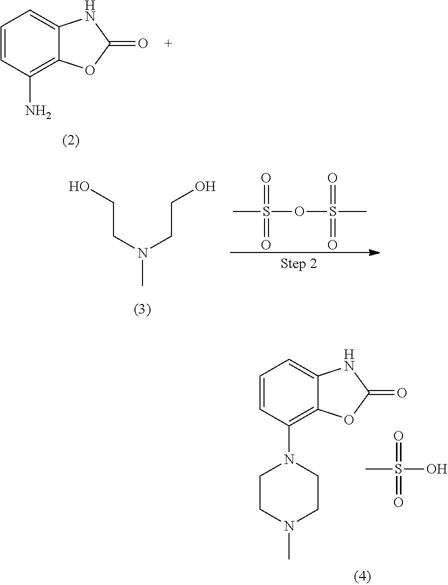

(iii) reacting 7-[(4-methyl)-1-piperazinyl]-2(3H)-benzoxazolone methanesulfonate (4) with a base, yielding 7-[(4-methyl)-1-piperazinyl]-2(3H)-benzoxazolone (5):

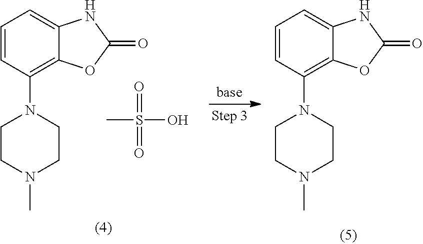

(iv) reacting 7-[(4-methyl)-1-piperazinyl]-2(3H)-benzoxazolone (5) with hydrochloric acid to yield 7-[(4-methyl)-1-piperazinyl]-2(3H)-benzoxazolone hydrochloride (6), as either the α-polymorph or the β-polymorph, depending on the conditions.

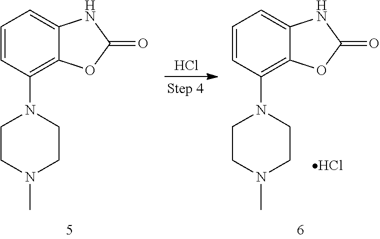

Up to and including 7-[(4-methyl)-1-piperazinyl]-2(3H)-benzoxazolone methanesulfonate (4), the synthetic steps can be performed as described in WO 02/066449.

The base used in step 3 can be chosen from alkaline compounds, such as sodium bicarbonate, potassium bicarbonate, sodium carbonate, potassium carbonate, alkaline hydroxides such as sodium hydroxide, potassium hydroxide or magnesium hydroxide, and alkaline phosphates such as dipotassium hydrogen phosphate. Also mixtures of these alkaline compounds can be used. In some embodiments, alkaline compounds are chosen from sodium bicarbonate, potassium bicarbonate, sodium carbonate, potassium carbonate and calcium carbonate. In other embodiments, the alkaline compound is sodium carbonate.

In order to synthesize the a-polymorph in step 4, the compound (5) is dissolved in a sufficient amount of a mixture of a polar solvent and water. Suitable polar solvents include acetonitrile, methyl ethyl ketone and isopropyl alcohol. In some embodiments, the polar solvent is acetonitrile.

The amount of water in the mixture in step 5 ranges approximately from 10% (w/w) to 30% (w/w). In order to dissolve compound (5), the mixture of the polar solvent and water is heated, for example, heated to reflux.

When the compound has been dissolved, HCl is added in an amount ranging from 1.05 to 1.45 molar equivalents (m/m) calculated based on the amount of compound (5) in the mixture. In some embodiments, the amount of HCl is 1.1 equivalents (m/m). In some embodiments, the HCl is added in the form of a concentrated solution in water, for example, the HCl is added as a 36% solution in water.

After the addition of HCl, for example, when a clear solution is obtained, the mixture is cooled to a temperature ranging from 25° C. to 0° C., for example, to approximately 0° C.

As soon as a crystalline product has been formed, the product can be isolated by a method known in the art as filtration or centrifugation.

After isolation the product can be dried, for example, at an elevated temperature and low pressure. In some embodiments, the drying temperature is from 20° C. to 70° C., for example, the drying temperature is 50° C. In some embodiments, the pressure during drying approximately ranges from 1,000 to 30 mbar. In some embodiments, the pressure during drying is approximately 100 mbar.

In order to synthesize the β-polymorph in step 4 the compound (5) is dissolved in a sufficient amount of a polar solvent. Suitable polar solvents include acetonitrile, methyl ethyl ketone and isopropyl alcohol. In some embodiments, the polar solvent can be acetonitrile.

In order to dissolve the compound (5) the polar solvent can be heated, for example, heated to reflux.

When the compound has been dissolved, HCl can be added in an amount ranging from 1.05 to 1.45 equivalents (m/m) calculated on the amount of compound (5) in the mixture. In some embodiments, the amount of HCl is 1.1 equivalents (m/m). In some embodiments, the HCl can be added in the form of a concentrated solution in water, for example, a 36% solution in water.

After the addition of HCl, for example, when a clear solution is obtained, the mixture can be cooled to a temperature ranging from 25° C. to 0° C., for example, approximately 0° C.

As soon as a crystalline product has been formed, the product can be isolated by a method known in the art, such as filtration or centrifugation.

After isolation, the product can be dried, for example, at elevated temperature and low pressure. In some embodiments, the drying temperature ranges from 20° C. to 70° C. In some embodiments, the drying temperature is 50° C. In some embodiments the pressure during drying ranges approximately from 1,000 to 30 mbar. For examples, the pressure during drying is about 100 mbar.

The compounds of the invention have interesting pharmacological properties, notably due to a combination of both partial dopamine D₂-receptor agonism and full serotonin 5-HT_{1A}-receptor agonism (WO 00/029397, Feenstra, 2001). Accordingly, the compounds are useful for treatment of conditionsor diseases of the central nervous system, caused by disturbances of the dopaminergic and/or serotonergic systems, for example: anxiety disorders (including generalized anxiety, panic disorder and obsessive compulsive disorder), depression, autism, schizophrenia, Parkinson's disease, restless leg syndrome, disturbances of cognition and memory.

Other embodiments of the invention include:

pharmaceutical compositions for treating, for example, a disorder or condition treatable by activating dopamine D₂ and/or serotonin 5-HT_{1A} receptors, the compositions comprising the α-polymorph of 7-[(4-methyl)-1-piperazinyl]-2(3H)-benzoxazolone hydrochloride, and a pharmaceutically acceptable carrier;

pharmaceutical compositions for treating a disorder or condition chosen from anxiety disorders (including generalised anxiety, panic disorder and obsessive compulsive disorder), depression, autism, schizophrenia, Parkinson's disease, restless leg syndrome, disturbances of cognition and memory, the pharmaceutical compositions comprising a compound of one of the embodiments of the invention, and a pharmaceutically acceptable carrier;

pharmaceutical compositions for treating a disorder or condition chosen from the disorders listed herein, the compositions comprising a compound of one of the embodiments of the invention, and a pharmaceutically acceptable carrier;

methods for treating a disorder or condition chosen from the disorders listed herein, the methods comprising administering to a patient in need of such treatment a compound of one of the embodiments of the invention. Embodiments of the invention also include the use of a compound of the invention for the manufacture of a medicament.

Other embodiments of the invention relate to combination therapies comprising a compound of the invention, or a pharmaceutical composition or formulation comprising a compound of one of the embodiments of the invention, administered concurrently or sequentially or as a combined preparation with another therapeutic agent or agents, for treating one or more of the conditions listed herein. Such other therapeutic agent(s) may be administered prior to, simultaneously with, or following the administration of the compounds of the invention.

### DEFINITIONS

To provide a more concise description, the terms ‘compound’ or ‘compounds’ include N-oxides, isotopically-labelled analogues, or pharmacologically acceptable salts, even when not explicitly mentioned.

‘Form’ is a term encompassing all solids: polymorphs, solvates, and amorphous forms. ‘Crystal form’ refers to various solid forms of the same compound, for example polymorphs, solvates and amorphous forms. ‘Amorphous forms’ are non-crystalline materials with no long range order, and generally do not give a distinctive powder X-ray diffraction pattern. Crystal forms in general have been described (Byrn et al., Pharmaceutical Research, 12(7), 945-954, 1995; Martin, E. W. (Editor), “Remington: The Science and Practice of Pharmacy”, Mack Publishing Company, 19^{th} Edition, Easton, Pa., Vol 2., Chapter 83, 1447-1462, 1995.).

Polymorphs' are crystal structures in which a compound can crystallize in different crystal packing arrangements, all of which have the same elemental composition. Polymorphism is a frequently occurring phenomenon, affected by several crystallization conditions such as temperature, level of supersaturation, the presence of impurities, polarity of solvent, rate of cooling. Different polymorphs usually have different X-ray diffraction patterns, solid state NMR spectra, infrared or Raman spectra, melting points, density, hardness, crystal shape, optical and electrical properties, stability, and solubility. Recrystallization solvent, rate of crystallization, storage temperature, and other factors may cause one crystal form to dominate.

To provide a more concise description, some of the quantitative expressions given herein are not qualified with either of the terms “about” or “approximately”. It is understood that whether either of the terms “about” or “approximately” is used explicitly or not, every quantity given herein is meant to refer to the actual given value, and it is also meant to refer to the approximation to such given value that would reasonably be inferred based on the ordinary skill in the art, including approximations due to experimental or measurement conditions for such given value.

Throughout the description and the claims of this specification the word “comprise” and variations of the word, such as “comprising” and “comprises” is not intended to exclude other additives, components, integers or steps.

While it may be possible for the compounds of the invention to be administered as the raw chemical, the compounds can also be administered as a ‘pharmaceutical composition’. According to a further aspect, embodiments of the present invention include a pharmaceutical composition comprising at least one compound of one of the embodiments of the invention, at least one pharmaceutically acceptable salt thereof, or a mixture of any of the foregoing, together with one or more pharmaceutically acceptable carriers thereof, and with or without one or more other therapeutic ingredients. The carrier(s) should be ‘acceptable’ in the sense of being compatible with the other ingredients of the formulation and not deleterious to the recipient thereof. The term “composition” as used herein encompasses a product comprising specified ingredients in predetermined amounts or proportions, as well as any product that results, directly or indirectly, from combining specified ingredients in specified amounts. In relation to pharmaceutical compositions, this term encompasses a product comprising one or more active ingredients, and an optional carrier comprising inert ingredients, as well as any product that results, directly or indirectly, from combination, complexation or aggregation of any two or more of the ingredients, or from dissociation of one or more of the ingredients, or from other types of reactions or interactions of one or more of the ingredients. In general, pharmaceutical compositions can be prepared by uniformly and intimately bringing the active ingredient into association with a liquid carrier or a finely divided solid carrier or both, and then, if necessary, shaping the product into the desired formulation. The pharmaceutical composition includes enough of the active object compound to produce the desired effect upon the progress or condition of diseases. Accordingly, the pharmaceutical compositions of the embodiments of the present invention encompass any composition made by admixing a compound of one of the embodiments of the present invention and a pharmaceutically acceptable carrier. By “pharmaceutically acceptable” it is meant the carrier, diluent or excipient is compatible with the other ingredients of the formulation and not deleterious to the recipient thereof.

Dose. The affinity of the compounds of the invention for dopamine D₂ and serotonin 5-HT_{1A} receptors was determined as described in WO 00/029397. From the binding affinity measured for a given compound of the embodiments of the invention, one can estimate a theoretical lowest effective dose. At a concentration of the compound equal to twice the measured Kᵢ-value, nearly 100% of the receptors will be occupied by the compound. By converting that concentration to mg of compound per kg of patient one obtains a theoretical lowest effective dose, assuming ideal bioavailability. Pharmacokinetic, pharmacodynamic, and other considerations may alter the dose actually administered to a higher or lower value. The typical daily dose of the active ingredients varies within a wide range and will depend on various factors such as the relevant indication, the route of administration, the age, weight and sex of the patient, and may be determined by a physician. In general, total daily dose administration to a patient in single or individual doses, may be in amounts, for example, from 0.001 to 10 mg/kg body weight daily, from 0.01 to 1,000 mg per day, or from 0.01 to 100 mg per day, of total active ingredients. Such dosages can be administered to a patient in need of treatment from one to three times each day, or as often as needed for efficacy, and for periods of at least two months, more typically for at least six months, or chronically.

The term “therapeutically effective amount” as used herein refers to an amount of a therapeutic agent useful to treat a condition treatable by administrating a composition of the invention. That amount includes the amount sufficient to exhibit a detectable therapeutic or ameliorative response in a tissue system or human. The effect may include, for example, treating the conditions listed herein. The precise pharmaceutically effective amount for a subject will depend upon the subject's size and health, the nature and extent of the condition being treated, recommendations of the treating physician, and the therapeutics, or combination of therapeutics, selected for administration. A “pharmaceutical salt” refers to an acid:base complex containing an active pharmaceutical ingredient (API) along with additional non-toxic molecular species in the same crystal structure. The term “pharmaceutically acceptable salt” refers to those salts that are, within the scope of sound medical judgment, suitable for use in contact with the tissues of humans without undue toxicity, irritation, allergic response, etc., and are commensurate with a reasonable benefit/risk ratio. Pharmaceutically acceptable salts are well-known in the art. They can be prepared in situ when finally isolating and purifying the compounds of the invention, or separately by reacting them with pharmaceutically acceptable non-toxic bases or acids, including inorganic or organic bases and inorganic or organic acids (Berge, S. M.: “Pharmaceutical salts”, J. Pharmaceutical Science, 66, 1-19 (1977)).

The ‘free base’ form may be regenerated by contacting the salt with a base or acid, and isolating the parent compound in the conventional matter. The parent form of the compound differs from the various salt forms in certain physical properties, such as solubility in polar solvents.

The term “treatment” as used herein refers to any treatment of a human condition or disease, and includes: (1) inhibiting the disease or condition, i.e., arresting its development, (2) relieving the disease or condition, i.e., causing the condition to regress, or (3) stopping the symptoms of the disease. The term Inhibit' includes its generally accepted meaning which includes restraining, alleviating, ameliorating, and slowing, stopping or reversing progression, severity, or a resultant symptom. As used herein, the term “medical therapy” includes diagnostic and therapeutic regimens carried out in vivo or ex vivo on humans.

### EXAMPLE 1: ANALYTICAL METHODS

X-ray Powder Diffraction (XRPD) patterns were measured on a diffractometer using CuKαᵢ radiation (tube voltage 40 kV, tube current 40 mA) at room temperature, using Bragg-Brentano geometry on a low background silicon wafer.

IR spectra were recorded on a Fourier transform IR spectrometer in attenuated total reflectance (diamond crystal) with a spectral resolution of 1 cm⁻¹ using a deuterated triglycine sulfate detector.

Raman spectra were recorded on a Fourier transform Raman spectrometer with a spectral resolution of 2 cm⁻¹ using a Ge diode detector. About 250 mW laser power was applied at an excitation wavelength of 1064 nm.

Single Crystal X-ray data were collected with a Nonius K-CCD diffractometer on a rotating anode at a temperature of 150 K, using MoKα radiation.

### EXAMPLE 2: SYNTHESES OF THE α- AND β-POLYMORPHS OF PARDOPRUNOX

Synthesis of 7-[(4-methyl)-1-piperazinyl]-2(3H)-benzoxazolone hydrochloride

Step 1: hydrogenation of 5-chloro-7-nitro-2(3H)-benzoxazolone (1) yielding 7-amino-2(3H)-benzoxazolone (2):

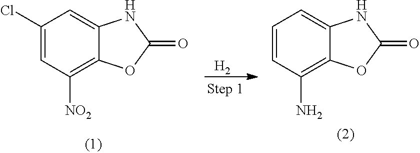

A suspension of 1.0 mol 5-chloro-7-nitro-2(3H)-benzoxazolone (1), 4.3 I ethanol, 150 ml ammonia 25% and 35 g Pd/C 10% was made at 60° C. This mixture was hydrogenated for 1 hour at 4 bar hydrogen pressure. The solution was cooled to 25° C. and filtered over hyflo. The solvent was changed to water and cooled to 0° C. The crystallized 7-amino-2(3H)-benzoxazolone (2) was isolated by filtration and washed with water/ethanol. The product was dried at 50° C. and 100 mbar to constant weight. The overall yield of this step was about 91% (crude on crude).

Step 2: construction of piperazine ring system by reacting 7-amino-2(3H)-benzoxazolone (2) with N-methyldiethanolamine (3) to yield 7-[(4-methyl)-1-piperazinyl]-2(3H)-benzoxazolone monomethanesulfonate (4).

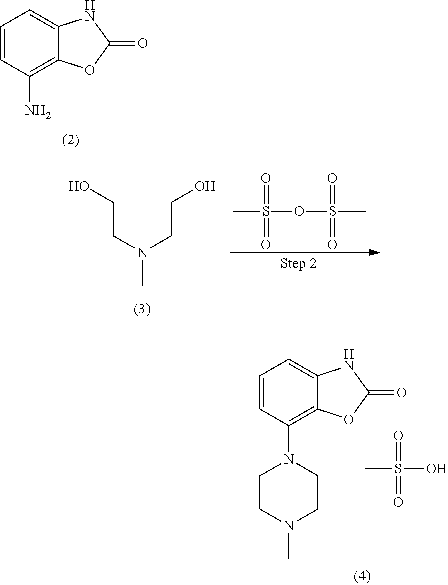

To a mixture of 14.9 g N-methyldiethanolamine (3), 44.5 g triethylamine and 120 ml methyl ethyl ketone (MEK) a mixture of 51.6 g methanesulfonic anhydride and 100 ml MEK was dosed at 0° C. Subsequently 14.5 g methanesulfonic acid was dosed at 0° C. After which, 14.5 g 7-amino-2(3H)-benzoxazolone (2) was added and the mixture was heated to reflux followed by a reflux period of 48 hours during which the product crystallized. The product was filtered off after cooling to 0° C. and washed with MEK. The product was dried at 50° C. and 100 mbar to constant weight. The overall yield of this step was about 67% (crude on crude).

Step 3: preparation of the free base from 7-[(4-methyl)-1-piperazinyl]-2(3H)-benzoxazolone monomethanesulfonate (4) to 7-[(4-methyl)-1-piperazinyl]-2(3H)-benzoxazolone (5):

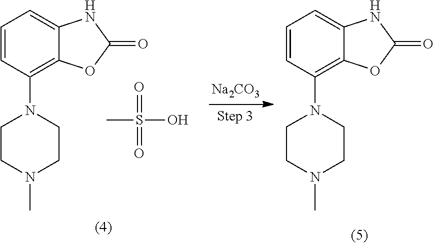

250 g of a 5% Na₂CO₃ solution was added to a mixture of 32.9 g 7-[(4-methyl)-1-piperazinyl]-2(3H)-benzoxazolone monomethanesulfonate (4) in 500 ml ethylacetate and stirred for 15 minutes at room temperature. The layers were separated and the water layer was washed three times with 150 ml ethylacetate. The ethylacetate layers were combined and the solvent was removed. 150 ml ethanol 96% was added to the residue at 50° C. The mixture was cooled to 0° C. and the product was isolated by filtration and washed with ethanol 96%. The product was dried at 50° C. and 100 mbar to constant weight. The overall yield of this step was about 90%.

Step 4: preparation of the hydrochloric acid salt of 7-[(4-methyl)-1-piperazinyl]-2(3H)-benzox-azolone (5) to 7-[(4-methyl)-1-piperazinyl]-2(3H)-benzoxazolone monohydrochloride (6)

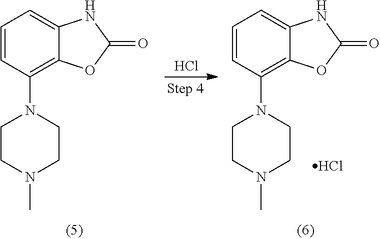

α-polymorph of 7-[(4-methyl)-1-piperazinyl]-2(3H)-benzoxazolone hydrochloride:

7-[(4-methyl)-1-piperazinyl]-2(3H)-benzoxazolone (5) was dissolved in sufficient amounts of a mixture of acetonitrile and water (90/10 w/w) to obtain a clear solution at reflux. 1.1 equivalent of 36% HCl was added at reflux. The mixture was cooled to 0° C. and the product was filtered off and washed with acetonitrile. The product was dried at 50° C. and 100 mbar to constant weight.

The overall yield of this step was about 91% (pure on crude).

β-polymorph of 7-[(4-methyl)-1-piperazinyl]-2(3H)-benzoxazolone hydrochloride:

7-[(4-methyl)-1-piperazinyl]-2(3H)-benzoxazolone (5) was dissolved in sufficient amounts of acetonitrile to obtain a clear solution at reflux. 1.1 equivalent of 36% HCl was added at reflux. The mixture was cooled to 0° C. and the product was filtered off and washed with acetonitrile. The product was dried at 50° C. and 100 mbar to constant weight. The overall yield of this step was about 100% (pure on crude).

### EXAMPLE 3: PHYSICOCHEMICAL PROPERTIES

The a and β-polymorphs were identified by single crystal X-Ray diffraction:

| Parameter: | α-polymorph | β-polymorph |
|---|---|---|
| temperature (°K) | 150 | 150 |
| wavelength (Å) | 0.71073 | 0.71073 |
| (Mo Kα radiation) | | |
| crystal system | monoclinic | monoclinic |
| space group | P21/c | C2/c |
| molecules per unit cell | 4 | 8 |
| Unit cell dimensions | | |
| a (Å) | 10.1685 | 23.958 |
| b (Å) | 13.995 | 7.2294 |
| c (Å) | 8.8323 | 16.625 |
| α (°) | 90 | 90 |
| β (°) | 91.66 | 120.528 |
| γ (°) | 90 | 90 |
| Calculated density (g cm⁻³) | 1.4260 | 1.4447 |
| Residual R-factor for structure | 2.86% | 4.05% |
| determination | | |

### EXAMPLE 4: STABILITY TESTS

Relative stability of the α- and β-polymorphs of pardoprunox were determined by ageing and slurry experiments in six different solvents. The crystal modification of the solid material was determined using XRPD. For mixtures, amounts of α and β were determined using semi-quantitative calculations, based on the ratio of peak heights of specific reflections of the α- and β-polymorphs, respectively. A peak at 23.3° 2θwas used for α-polymorphs, and one at 15.3° 2θfor β-polymorphs. Due to effects of sample preparation, crystal orientation and differences in response factors, this estimation is semi-quantitative.

Ageing experiments

For ageing experiments, two series of saturated solutions of a specific polymorphic (α or β) form were shaken at 375 rpm, for one week, in six different solvents, one series at ambient temperature (ca. 20° C.) and one at 50° C. 40 ml tubes were filled with 0.5 g of the appropriate polymorphic form, and 25 ml of solvent (or mixture). After a week the precipitate was filtered and dried at ambient temperature under reduced pressure. The results of the ageing experiments for α- and β-polymorphs are given in Table 1.

TABLE 1
Ageing experiments of α- and β-polymorphs of pardoprunox

| | | Crystal modifications by XRPD | |
|---|---|---|---|
| Solvent (mixture) | temp | α-polymorph | β-polymorph |
| 96% ethanol | ambient | α (100%) | α |
| acetonitrile | ambient | α | α (18%) + β (82%) |
| methyl ethyl ketone | ambient | α | α (4%) + β (96%) |
| ethyl acetate/isopropanol | ambient | α | α (3%) + β (97%) |
| (2:1) | | | |
| 1,2-dimethoxy ethane | ambient | α | α (7%) + β (93%) |
| toluene/methanol (10:3) | ambient | α | α |
| 96% ethanol | 50° C. | α | α |
| acetonitrile | 50° C. | α | α (62%) + β (38%) |
| methyl ethyl ketone | 50° C. | α | α (5%) + β (95%) |
| ethyl acetate/isopropanol | 50° C. | α | α (8%) + β (92%) |
| (2:1) | | | |
| 1,2-dimethoxy ethane | 50° C. | α | α (8%) + β (92%) |
| toluene/methanol (10:3) | 50° C. | α | α |

Slurry experiments

For slurry experiments, two series of saturated solutions of a specific polymorphic (α or β) form were shaken at 375 rpm for one day in six different solvents, one series at ambient temperature and one at 50° C. 40 ml tubes were filled with 0.5 g of the appropriate polymorphic form, and 25 ml of solvent (or mixture). After 1 day about 2.5 ml of sample was taken from each tube, filtered and dried at ambient temperature under reduced pressure. Subsequently the crystal modification was determined. After the samples were taken each tube was seeded with 15-20 mg of the other polymorphic form. Then, all tubes were shaken for 1 week at 375 rpm at ambient temperature or at 50° C. Finally, the precipitate was filtered and dried at ambient temperature under reduced pressure and the crystal modification was determined. The results of the slurry experiments for α- and β-polymorphs are given in Table 2.

TABLE 2
Slurry experiments of α- and β-polymorphs of pardoprunox

| | | Crystal modifications by XRPD | | |
|---|---|---|---|---|
| | | | β-polymorph | |
| Solvent (mixture) | temp | α-polymorph | before seeding | After seeding |
| 96% ethanol | ambient | α (100%) | α | α |
| acetonitrile | ambient | α | α(4%) + β(96%) | α(44%) + β(56%) |
| methyl ethyl ketone | ambient | α | α(6%) + β(94%) | α(8%) + β(92%) |
| ethyl acetate/isopropanol (2:1) | ambient | α | α(4%) + β(96%) | α(6%) + β(94%) |
| 1,2-dimethoxy ethane | ambient | α | α(4%) + β(96%) | α(10%) + β(90%) |
| toluene/methanol (10:3) | ambient | α | α(39) + β(61%) | α |
| 96% ethanol | 50° C. | α | α | α |
| acetonitrile | 50° C. | α | α(14%) + β(86%) | α |
| methyl ethyl ketone | 50° C. | α | α(9%) + β(91%) | α(11%) + β(89%) |
| ethyl acetate/isopropanol (2:1) | 50° C. | α | α(8%) + β(92%) | α(10%) + β(90%) |
| 1,2-dimethoxy ethane | 50° C. | α | α(6%) + β(94%) | α(13%) + β(87%) |
| toluene/methanol (10:3) | 50° C. | α | α | α |

Neither in ageing nor in slurry experiments, was conversion from α- to β-polymorph observed.

In ageing as well as in slurry experiments, both at ambient temperature and at 50° C., a complete conversion from β- to α-polymorph was observed in ethanol and in a 10:3 toluene/methanol mixture; in acetonitrile substantial conversion was observed, while in other solvents the conversion was minimal.

These results demonstrate that crystal modification a is more stable than crystal modification β at the applied experimental conditions.

### EXAMPLE 5: PHARMACEUTICAL PREPARATIONS

For clinical use, a compound of the invention is formulated into a pharmaceutical composition, which is a novel embodiment of the invention because it contains a novel compound disclosed herein. Types of pharmaceutical compositions that may be used include: tablets, chewable tablets, capsules (including microcapsules), solutions, parenteral solutions, ointments (creams and gels), suppositories, suspensions, and other types disclosed herein, or are apparent to a person skilled in the art from the specification and general knowledge in the art. The active ingredient may also be in the form of an inclusion complex in cyclodextrins, their ethers or their esters. The compositions are used for oral, intravenous, subcutaneous, tracheal, bronchial, intranasal, pulmonary, transdermal, buccal, rectal, parenteral or other ways to administer. The pharmaceutical formulation contains the compound of the invention in admixture with at least one pharmaceutically acceptable adjuvant, diluent and/or carrier. In embodiments of the present invention, the total amount of active ingredient can be in the range of from about 0.1% (w/w) to about 95% (w/w) of the formulation, such as from 0.5% to 50% (w/w) and from 1% to 25% (w/w). In some embodiments, the amount of active ingredient can be greater than about 95% (w/w) or less than about 0.1% (w/w).

The compound of the invention can be brought into forms suitable for administration by means of usual processes using auxiliary substances such as liquid or solid, powdered ingredients, such as the pharmaceutically customary liquid or solid fillers and extenders, solvents, emulsifiers, lubricants, flavorings, colorings and/or buffer substances. Frequently used auxiliary substances include magnesium carbonate, titanium dioxide, lactose, saccharose, sorbitol, mannitol and other sugars or sugar alcohols, talc, lactoprotein, gelatin, starch, amylopectin, cellulose and its derivatives, animal and vegetable oils such as fish liver oil, sunflower, groundnut or sesame oil, polyethylene glycol and solvents such as, for example, sterile water and mono- or polyhydric alcohols such as glycerol, as well as with disintegrating agents and lubricating agents such as magnesium stearate, calcium stearate, sodium stearyl fumarate and polyethylene glycol waxes. The mixture may then be processed into granules or pressed into tablets. A tablet can be prepared using the ingredients below:

| Ingredient | Quantity (mg/tablet) |
|---|---|
| α-polymorph of pardoprunox | 10 |
| Cellulose, microcrystalline | 200 |
| Silicon dioxide, fumed | 10 |
| Stearic acid | 10 |
| Total | 230 |

The components are blended and compressed to form tablets each weighing 230 mg. The active ingredient may be separately premixed with the other non-active ingredients, before being mixed to form a formulation.

Soft gelatin capsules may be prepared with capsules containing a mixture of the active ingredients of the invention, vegetable oil, fat, or other suitable vehicle for soft gelatin capsules. Hard gelatin capsules may contain granules of the active ingredients. Hard gelatin capsules may also contain the active ingredients together with solid powdered ingredients such as lactose, saccharose, sorbitol, mannitol, potato starch, corn starch, amylopectin, cellulose derivatives or gelatin.

Dosage units for rectal administration may be prepared (i) in the form of suppositories that contain the active substance mixed with a neutral fat base; (ii) in the form of a gelatin rectal capsule that contains the active substance in a mixture with a vegetable oil, paraffin oil or other suitable vehicle for gelatin rectal capsules; (iii) in the form of a ready-made micro enema; or (iv) in the form of a dry micro enema formulation to be reconstituted in a suitable solvent just prior to administration.

Liquid preparations may be prepared in the form of syrups, elixirs, concentrated drops or suspensions, e.g. solutions or suspensions containing the active ingredients and the remainder consisting, for example, of sugar or sugar alcohols and a mixture of ethanol, water, glycerol, propylene glycol and polyethylene glycol. If desired, such liquid preparations may contain coloring agents, flavoring agents, preservatives, saccharine and carboxymethyl cellulose or other thickening agents. Liquid preparations may also be prepared in the form of a dry powder, reconstituted with a suitable solvent prior to use. Solutions for parenteral administration may be prepared as a solution of a formulation of the invention in a pharmaceutically acceptable solvent. These solutions may also contain stabilizing ingredients, preservatives and/or buffering ingredients. Solutions for parenteral administration may also be prepared as a dry preparation, reconstituted with a suitable solvent before use.

Also provided according to the present invention are formulations and ‘kits of parts’ comprising one or more containers filled with one or more of the ingredients of a pharmaceutical composition of the invention, for use in medical therapy. Associated with such container(s) can be various written materials such as instructions for use, or a notice in the form prescribed by a governmental agency regulating the manufacture, use or sale of pharmaceuticals products, which notice reflects approval by the agency of manufacture, use, or sale for human administration. The use of formulations of the present invention in the manufacture of medicaments for use in treating a condition in which activation of dopamine D₂ and/or serotonin 5-HT_{1A} receptors is required or desired, and methods of medical treatment, comprise the administration of a therapeutically effective total amount of at least one compound of the invention to a patient suffering from, or susceptible to, a condition in which activation of dopamine D₂ and/or serotonin 5-HT_{1A} receptors is required or desired.

What is claimed is:

1. The α-polymorph of 7-[(4-methyl)-1-piperazinyl]-2(3H)-benzoxazolone hydrochloride, wherein the polymorph exhibits an X-ray powder diffraction pattern having characteristic reflexes, expressed in degrees of diffraction angle 2 θ, at about: 17.4, 21.5, 23.3, and 28.8, an infrared spectrum recorded in Attenuated Total Reflectance having characteristic absorption bands expressed in reciprocal centimeters at about: 2454 and 1604, and a Raman spectrum having characteristic absorption bands expressed in reciprocal centimeters at about: 3079, 3031 and 1632.

2. The polymorph claimed in claim 1, wherein the polymorph exhibits an X-ray powder diffraction pattern having characteristic reflexes, expressed in degrees of diffraction angle 2 θ, at about: 15.3, 17.4, 18.4, 20.1, 20.9, 21.5, 23.3, 23.6, 25.4, and 28.8, an infrared spectrum recorded in Attenuated Total Reflectance having characteristic absorption bands expressed in reciprocal centimeters at about: 2454, 1749, 1632, 1604, 1456, 1394, 1265, 1144, 947, and 735, and a Raman spectrum having characteristic absorption bands expressed in reciprocal centimeters at about: 3079, 3031, 2987, 2972, 1632, 1262, 859, 561, 499, and 273.

3. A pharmaceutical composition comprising, in addition to a pharmaceutically acceptable carrier and at least one pharmaceutically acceptable auxiliary substance, a pharmacologically active amount of the α-polymorph of 7-[(4-methyl)-1-piperazinyl]-2(3H)-benzoxazolone hydrochloride, as an active ingredient, wherein the polymorph exhibits an X-ray powder diffraction pattern having characteristic reflexes, expressed in degrees of diffraction angle 2 θ, at about: 17.4, 21.5, 23.3, and 28.8, an infrared spectrum recorded in Attenuated Total Reflectance having characteristic absorption bands expressed in reciprocal centimeters at about: 2454 and 1604, and a Raman spectrum having characteristic absorption bands expressed in reciprocal centimeters at about: 3079, 3031 and 1632.

4. The pharmaceutical composition as claimed in claim 3, wherein the polymorph exhibits an X-ray powder diffraction pattern having characteristic reflexes, expressed in degrees of diffraction angle 2 θ, at about: 15.3, 17.4, 18.4, 20.1, 20.9, 21.5, 23.3, 23.6, 25.4, and 28.8, an infrared spectrum recorded in Attenuated Total Reflectance having characteristic absorption bands expressed in reciprocal centimeters at about: 2454, 1749, 1632, 1604, 1456, 1394, 1265, 1144, 947, and 735, and a Raman spectrum having characteristic absorption bands expressed in reciprocal centimeters at about: 3079, 3031, 2987, 2972, 1632, 1262, 859, 561, 499, and 273.

5. A method for treating at least one central nervous system disorder chosen from anxiety disorders, depression, autism, schizophrenia, Parkinson's disease, restless leg syndrome, and disturbances of cognition and memory, the method comprising administering a pharmaceutical composition to a patient in need thereof, said composition comprising the α-polymorph of 7-[(4-methyl)-1-piperazinyl]-2(3H)-benzoxazolone hydrochloride, wherein the polymorph exhibits an X-ray powder diffraction pattern having characteristic reflexes, expressed in degrees of diffraction angle 2 θ, at about: 17.4, 21.5, 23.3, and 28.8, an infrared spectrum recorded in Attenuated Total Reflectance having characteristic absorption bands expressed in reciprocal centimeters at about: 2454 and 1604, and a Raman spectrum having characteristic absorption bands expressed in reciprocal centimeters at about: 3079, 3031 and 1632.

6. The method as claimed in claim 5, wherein the polymorph exhibits an X-ray powder diffraction pattern having characteristic reflexes, expressed in degrees of diffraction angle 2 θ, at about: 15.3, 17.4, 18.4, 20.1, 20.9, 21.5, 23.3, 23.6, 25.4, and 28.8, an infrared spectrum recorded in Attenuated Total Reflectance having characteristic absorption bands expressed in reciprocal centimeters at about: 2454, 1749, 1632, 1604, 1456, 1394, 1265, 1144, 947, and 735, and a Raman spectrum having characteristic absorption bands expressed in reciprocal centimeters at about: 3079, 3031, 2987, 2972, 1632, 1262, 859, 561, 499, and 273.

7. A method for preparing an α-polymorph of 7-[(4-methyl)-1-piperazinyl]-2(3H)-benzoxazolone hydrochloride, comprising:
(i) dissolving 7-[(4-methyl)-1-piperazinyl]-2(3H)-benzoxazolone (5) in a mixture of a polar solvent and water; 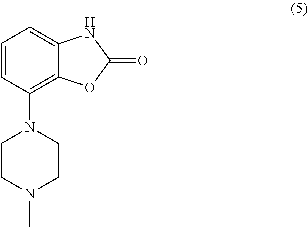
(ii) adding HCl; and
(iii) isolating the product as a crystalline product.

8. The method as claimed in claim 7, wherein said polar solvent is chosen from acetonitrile, methyl ethyl ketone, and isopropylalcohol.

9. The method as claimed in claim 8, wherein the polar solvent is acetonitrile.

10. The method as claimed in claim 7, wherein said mixture comprises from 10% (w/w) to 30% (w/w) water.

11. The method as claimed in claim 7, wherein from 1.05 to 1.45 equivalents of HCl is added in step (ii).

12. The method as claimed in claim 11, wherein the HCl is in the form of 36% hydrochloric acid in water.
